(19) United States
(12) Patent Application Publication
(10) Pub. No.: US 2025/0224367 A1
(43) Pub. Date: Jul. 10, 2025

(54) BIOSENSORS USING CARBON NANOTUBES AND AN ELECTRONIC READER FOR USE WITH THE BIOSENSORS

(71) Applicant: HeMemics Biotechnologies, Inc., Gaithersburg, MD (US)

(72) Inventors: David Ho, Mclean, VA (US); John L. Warden, JR., Chevy Chase, MD (US)

(21) Appl. No.: 18/802,925

(22) Filed: Aug. 13, 2024

Related U.S. Application Data
(63) Continuation of application No. 17/282,824, filed on Apr. 5, 2021, now abandoned.
(60) Provisional application No. 62/741,980, filed on Oct. 5, 2018.
(60) Provisional application No. 62/777,504, filed on Dec. 10, 2018.

(51) Int. Cl. G01N 27/414; G01N 33/543; G01N 33/569

(52) U.S. Cl. CPC G01N 27/4146; G01N 27/4145; G01N 33/54373; G01N 33/56916; G01N 2333/245

(57) ABSTRACT

Disclosed herein are biosensors, and, more particularly, biosensors using carbon nanotubes, an electronic reader for use with the biosensors, and systems and methods employing them. The biosensors employ preserved biologics on the carbon nanotubes which results in shelf-stable, robust biosensors.

### CROSS REFERENCE TO RELATED APPLICATIONS

This application claims the benefit of priority of U.S. Provisional Patent Application Ser. No. 62/741,980, filed on Oct. 5, 2018, and U.S. Provisional Patent Application Ser. No. 62/777,504, filed on Dec. 10, 2018, the entire contents of each of which is hereby incorporated by reference herein.

### TECHNICAL FIELD

The present disclosure relates to biosensors, and, more particularly, biosensors using carbon nanotubes, an electronic reader for use with the biosensors, and systems and methods employing them.

### BACKGROUND

Biosensor technology is a growing technological field. Current sensing technology for the presence of pathogens such as bacteria is generally limited to visual tests that result from applying a sample to a test strip and observing a change over a period of time (e.g., on the magnitude of several minutes or hours). Other testing options include manual inspection of samples under microscope. For example, many biomarker detection methods are based on optical detection of a ligand, either directly as in the case of high-performance liquid chromatography, or by secondary means such as through a labeled detector antibody in the enzyme-linked immunosorbent assay (ELISA).

A further biomarker detection method is in the form of carbon nanotube (CNT) biosensors, which are based on the electronic detection of a ligand. A CNT biosensor is a semiconductor element effectively having three terminals; a source, a drain, and a gate electrode, a configuration similar to that of a conventional metal-oxide-semiconductor field-effect transistor. However, the broad application of carbon nanotubes in biomedicine is hindered by their solid or semisolid properties. Therefore, experimental efforts have been made by functionalizing CNTs with biological materials to make CNTs soluble or capable of being applied in various forms such as biosensors, gene and drug delivery.

Changes in structure, size, surface chemistry, charge, and shape are key parameters in determining the bioactivity of immobilized proteins. The activity and exquisite selectivity of proteins, such as antibodies, requires the near complete retention of the native structure. When CNTs interact with proteins, certain binding selectivity has been observed. Solid or semisolid interactions, especially π-π stacking interactions and electrostatic interactions are reported to play key roles in CNT/protein binding. Protein conformational changes and protein partial denaturation all promote CNT/protein binding. This suggests that the more denatured solid or semisolid protein interior has a greater propensity to bind to CNTs and the structure/function, and spatial orientation of proteins adsorbed onto carbon nanotubes has been shown to be greatly altered.

Carbon Nanotube Field Effect Transistor (CNT-FET) biosensors are one exemplary implementation of CNTs in a device to detect a target biological substance. In most cases, the CNT-FET relies on having biological materials such as cells, antibodies and other macromolecules binding components maintain their viability and native structures for extended periods whilst being immobilized onto carbon nanotubes. However, the carbon nanotubes are extremely solid or semisolid and biologics do not retain their activities when immobilized on these types of surfaces. Thus, methods of stabilizing the biologics on a CNT and methods and systems for employing them as biosensors are needed. Moreover, there is a lack of accuracy that may lead to less that desirable accuracy and precision.

The present disclosure is directed to overcoming these and other problems associated with the prior art.

### SUMMARY

Some embodiments provide a biosensor for producing a signal indicative of the presence of at least one target biologic, the biosensor comprising an electrical connector configured to connect to a electrical circuit and produce an output signal; and an active area comprising a material deposited on a base material between two or more electrodes connected to the electrical connector, wherein the material comprises a plurality of carbon nanotubes; a detecting biologic; and a preservative solution, wherein an impedance value of the output signal is dependent on the detecting biologic and the presence of the target biologic.

In some embodiments, each electrical connector comprises a plurality of electrical contacts.

In some embodiments, the detecting biologic is an antibody and the target biologic is an antigen.

In some embodiments, the detecting biologic is an antigen and the target biologic is an antibody.

In some embodiments, the detecting biologic is one or more of a carbohydrate, lipid, protein, nucleic acid, whole cell, cell fragment, prokaryotic cell, parasite, virus, nucleated or enucleated cell, or intercellular organelle. The detecting biologic may be a chemical or reagent according to some embodiments.

In some embodiments, the preservative solution comprises at least one sugar with molecular weight larger than 40,000 Da and at least another sugar with molecular weight of less than 40,000 Da. In some embodiments, wherein the moisture content of the material on the base material is from 5% to 95%.

In some embodiments, further comprise a processing area for storing information about the biosensor.

In some embodiments, the processing area comprises an RFID tag and an antenna.

In some embodiments, the stored information includes one or more of serial number, batch number, date information, and encrypted data for tamper-proof security.

In some embodiments, the base material is a silicon solid or semi-solid substrate.

Some embodiments provide a biosensor system for detecting the presence of a target biologic, the system comprising an electronic reader comprising a circuit for delivering a signal and a processing device for reading the signal; and a biosensor for operative connection to the electronic reader, the biosensor comprising: an electrical connector configured to connect to a electrical circuit and produce an output signal; and an active area comprising a material deposited on a base material between two or more electrodes connected to the electrical connector, wherein the material comprises: a plurality of carbon nanotubes; a detecting biologic; and a preservative solution, wherein the electronic reader is configured to apply the signal to the biosensor and obtain an output impedance value before and after a sample has been applied to the active area, and compare the output impedance values to determine whether a binding event has occurred as a result of the sample being applied to the active area.

In some embodiments, the detecting biologic is an antibody and the target biologic is an antigen.

In some embodiments, the detecting biologic is an antigen and the target biologic is an antibody.

In some embodiments, the detecting biologic is one or more of a carbohydrate, lipid, protein, nucleic acid, whole cell, cell fragment, prokaryotic cell, parasite, virus, nucleated or enucleated cell, or intercellular organelle. The detecting biologic may be a chemical or reagent according to some embodiments.

In some embodiments, the circuit comprises an RC circuit configured to deliver a pulsed signal.

Some embodiments provide a method of detecting a target biologic using a biosensor and an electronic reader, comprising: electrically connecting the biosensor to the electronic reader; applying an AC signal to the biosensor via a circuit in the electronic reader; determining a base resistance between a pair of electrodes on the biosensor; identifying a change in resistance based on a binding event between a detecting biologic on the biosensor and a target biologic in a sample applied to the biosensor; outputting a detection decision based on the identified change in resistance.

In some embodiments, the base resistance is determined prior to a sample being applied to the biosensor.

In some embodiments, the electronic reader applies a signal pulse to the biosensor to identify the change in resistance.

In some embodiments, the electronic reader applies a voltage between 1-3 V in the AC signal.

In some embodiments, the target biologic is conjugated to a particle, such as a gold nanoparticle.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 is a schematic diagram of an exemplary biosensor using carbon nanotubes, consistent with disclosed embodiments;

FIG. 2 is a top view of an exemplary biosensor in the form a biosensor chip, consistent with disclosed embodiments;

FIG. 3 is a perspective view of an exemplary electronic reader for use in combination with the biosensor chip of FIG. 2, consistent with disclosed embodiments;

FIG. 4 is a circuit diagram further illustrating an exemplary circuit that may be used in combination with the electronic reader of FIG. 3, consistent with disclosed embodiments;

FIG. 5 is a flowchart of an exemplary process for detecting a target biologic using a biosensor chip and electronic reader, consistent with disclosed embodiments;

FIG. 6 is a table that lists various proteins and formats along with the extended shelf-life after preservation, consistent with disclosed embodiments;

FIG. 7A is a depiction of a graph illustrating an exemplary signal that may be detected by the electronic reader of FIG. 3, consistent with disclosed embodiments;

FIG. 7B is a depiction of another graph illustrating an exemplary signal that may be detected by the electronic reader of FIG. 3, consistent with disclosed embodiments;

FIG. 8A is a depiction of an exemplary embodiment of a binding event in an embodiment including labeled detection of a target biologic;

FIG. 8B is another depiction of an exemplary embodiment of a binding event in an embodiment including labeled detection of a target biologic

FIG. 9 is a table that lists several bacteria and conditions tested using the exemplary sample carrier and electronic reader, consistent with disclosed embodiments; and

FIG. 10 shows exemplary concentration measurements, consistent with disclosed embodiments.

### DETAILED DESCRIPTION OF THE EXEMPLARY EMBODIMENTS

The present disclosure relates to biosensors for detecting a target material, an electronic reader for use in combination with the biosensors, and systems and methods employing them. The biosensors include carbon nanotubes in combination with biologics on the surface of the CNTs. The biosensors, in an embodiment, may be configured as a chip insertable into the electronic reader for determining the presence or absence of a detectable material, such as an antigen, antibody, or other biologic substance. The electronic reader may include a circuit configured to detect a signal through the chip and analyze the signal to determine the presence or absence of the detectable material.

Consistent with disclosed embodiments, a biosensor chip may be configured to detect the presence of a target material through a paired detection material. According to at least some embodiments, the target material and detection material are pairs that interact with each other through at least one of a mechanical, electrical, or chemical process. For instance, the disclose embodiments may use paired biologics, such as an antigen-antibody pair that conjugate with each other when combined. The term biologic is used herein to describe a material, molecule, or other particle or combination of particles such as, but not limited to, carbohydrates, lipids, proteins, and nucleic acids (DNA, RNA), recombinant proteins, hybrid molecules such as protein conjugated to DNA or RNA, DNA conjugated to carbohydrates, chimera and others, etc. The term biologic may also include, for example, whole cells or cell fragments of mammalian cells, prokaryotic cells, parasites, viruses, nucleated or enucleated cells. Further embodiments of biologics that may be used in conjunction with the disclosed embodiments include intracellular organelles (e.g., ribosomes, endoplasmic reticulum, nuclear envelope), and/or chemicals and reagents such as antibiotics, drugs, inorganic molecules, synthetic molecules, and organic molecules.

An exemplary biosensor chip may be configured with an electronic interface for connecting to the circuit of the electronic reader. The biosensor chip further includes an active area including a material deposited on a substrate between at least two electrodes. For examples, the biosensor chip may include a CNT-FET configuration for detecting a signal across a gap between the electrodes via the deposited material. The material may include carbon nanotubes, a preservative solution, and a detecting biologic. The biosensor chip may thus be configured to provide a testing result when a sample (e.g., blood, saliva, skin, etc.) is applied based on a reaction between a target biologic in the sample and the detecting biologic. For instance, the detecting biologic may be an antibody configured to bind to a target biologic in the form of a particular antigen present in the sample.

Disclosed embodiments also include the use of a preservative solution to stabilize a biologic substance on a solid surface (e.g., a silicon substrate) in combination with the CNTs. CNTs are hollow graphitic nanomaterials that have a very ordered structure and are ultra-light weight. CNTs also have excellent mechanical strength, electrical and thermal conductivity, and can have both metallic and semiconductor behavior. Combining the biologic substance with polymers and graphene added to carbon nanotubes can produce a CNT-FET. Together, the dry stabilized biologics (i.e. detecting biologic) bind to CNT-FET with an extended lifetime that enable them to be used as a biosensor.

The rapid development of carbon nanotube technology has already had a major impact on the material science and nanoelectronics fields and will undoubtedly enhance the performance of veterinary diagnostic assays and instrumentation. The exciting potential for using carbon nanotube technology in medical diagnostics is that it opens up a new dimension for biomarker detection.

Since biologics such as antibodies are unstable when immobilized on surfaces such as CNTs, the present disclosure describes compositions including desiccated biologics comprising cells, bacteria, viruses, protein, nucleic acid, lipids, carbohydrate, or any combination thereof, stabilized on a substrate for use as a biosensor. In some embodiments, the material deposited on a disclosed biosensor chip further includes at least one sugar, such as a sugar with molecular weight larger than 40,000 Da, and at least another sugar with MS of less than 40,000 Da. In disclosed embodiments, the moisture content of the biologics on the solid or semisolid surface is from 5% to 95%.

A disclosed method of preserving biologics onto surfaces may include immobilizing the biologics onto a surface via non-covalent and/or covalent attachment; contacting the biologics with a preservative solution comprising at least one large MW sugar (>40,000 Da) and at least another smaller MW sugar (<40,000 Da); and drying the biologics to a final moisture content of from about 5% to about 95%. At least one large MW sugar and the at least another small MW sugar can be present in a single preservative solution or may be separate solutions. These preserving liquids and methods are described in U.S. Pat. Nos. 8,628,960, 9,642,353, and 9,943,075, each of which is incorporated herein in its entirety.

In some embodiments, the detecting biologics that are immobilized on the solid or semisolid surfaces include, for example, antibodies, cells, bacteria, viruses, protein, nucleic acid, lipids, carbohydrate, or any combination thereof. In some embodiments, the target biologics include, for example, antigens, cells, bacteria, viruses, protein, nucleic acid, lipids, carbohydrate, or any combination thereof. In some embodiments, the configuration may be reversed and, for example, an antigen may be bonded to a biosensor chip for detecting an antibody in a sample.

In some embodiments, the solid or semisolid surface is provided either alone or in combination with: plastic such as polyethylene, polypropylene, polystyrene or the like; metal such as Au, Ag or the like; semiconductor such as CdS, CdSe or the like; magnetics such as Fe₃O₄ or the like; carbon based such as fullerene, carbon nanotubes, graphene or the like. In some embodiments, the substrate is provided in combination with semisolid surfaces such as agar, gelatin, cream, ointment or the like. In some instances, the substrate includes a surface that is hydrophobic, such as in the case of carbon nanotubes, graphene, and the like.

In some embodiments, the preservative solution included in the deposited material comprises at least one membrane penetrable sugar, at least one membrane impenetrable sugar, at least one anti-microbial agent, at least one anti-oxidant, optionally a salt, adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), optionally adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), adenosine, albumin, a salt (e.g., chloride salts such as KCl, NaCl, CaCl₂, and covalent chlorides of metals or nonmetals such as titanium(IV) chloride or carbon tetrachloride), a buffer (e.g., K₂HPO₄), and a chelating agent (e.g., EDTA).

Use of the preservative solution allows the preservation of one or more biologics in the absence of lyophilization, freeze-drying, vacuum drying, and/or oven-drying steps. In some embodiments, the methods comprise contacting or suspending one or more biologics with a preservative solution. To preserve the biologic material (e.g., the detecting biologic), for example an antibody, the preservative solution is prepared, in some instances by making a dilution of preservative solution in a buffer. The biologic is prepared or reconstituted, as necessary, in accordance with its manufacturer's instructions.

In an exemplary embodiment, the preservative solution is a solution as described in U.S. Pat. No. 9,943,075, hereby incorporated by reference in its entirety and co-owned by Applicant. U.S. Pat. No. 9,943,075 describes a preservative solution useful in the embodiments described herein. In the descriptions below and in the examples, a commercial product, HemSol™, was used as the preservative solution. In one example, a user may produce a preservative solution for antibody by making 1:100 to 1:500 dilution of HemSol™ in phosphate buffered saline (PBS) or similar buffer—this is the HemSol™ antibody buffer to dilute and preserve antibody.

The biosensor chip, including the deposited material having a detecting biologic, CNT-FET, and preservative solution, provides a biosensor that has a long shelf-life with high reliability. Difficulty in maintaining detecting biologics on a substrate is addressed by the preservative solution, which enables the detecting biologics to maintain their form for an extended period of time, and at room temperature. Thus, unlike current sensing technologies, the biosensors can be made in bulk and stored until use. The biosensor chip configuration provides an easy-to-use and effective system for detecting a target biologic via an electronic detection method, where the biosensor chips may be manufactured in large quantities for ready availability. Moreover, the configuration of a layered substrate for providing a plurality of CNT-FET sites enables simultaneous testing for multiple target biologics. As a result, a robust biosensor device is formed for reliable, fast, and comprehensive detection of multiple target biologics in a single sample (e.g., drop of blood, saliva or nucleic acid, etc.).

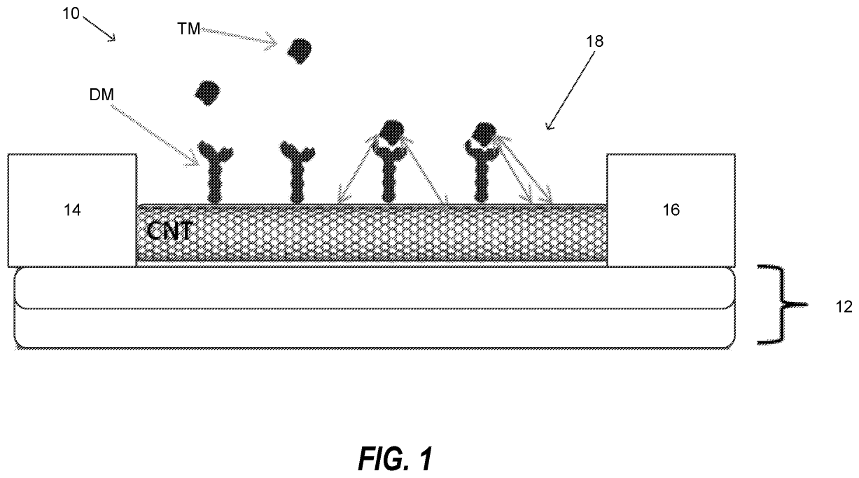

FIG. 1 is a schematic illustration of an exemplary embodiment of a biosensor 10. The biosensor 10 includes a substrate 12, such as a silica substrate. The biosensor 10 further includes a circuit formed on the substrate 12, the circuit having at least two electrodes, such as a source electrode 14 and a drain electrode 16. The biosensor 10 further includes a material 18 between the electrodes. The material 18 may include at least a carbon nanotube portion, a preservative solution, and a detecting biologic (DM). The carbon nanotube portion may include CNTs and graphene.

The biosensor 10 may be configured for detection of a target biologic (TM) through a binding event with the detecting biologic in the deposited material 18. For example, when a sample is applied to the biosensor 10, a target biologic, such as an antigen, may bind to a detecting biologic in the form of an antibody. The resulting binding event effects the impedance of the carbon nanotube portion. Thus, by monitoring a signal across the electrodes 14, 16, it is possible to determine the presence and/or absence of a target biologic (e.g., the antigen).

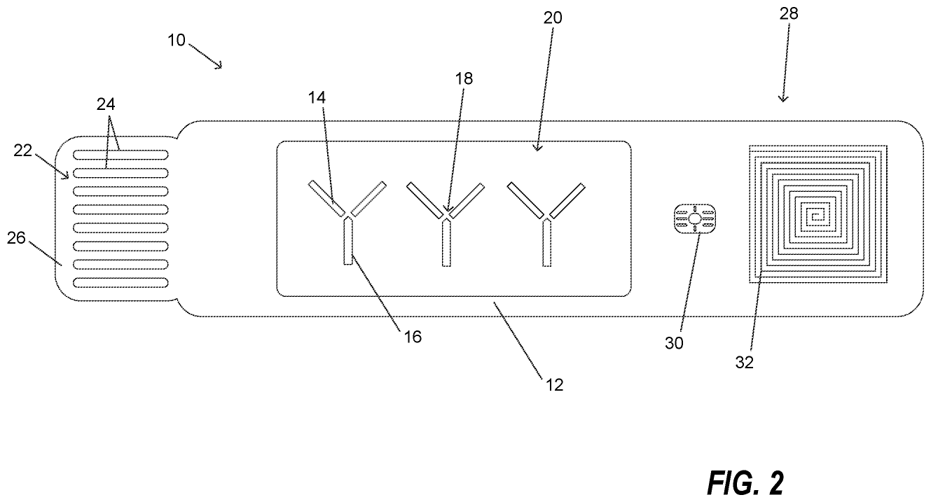

FIG. 2 is a top view of the biosensor 10. The biosensor 10 includes an active area 20. The active area 20 includes the substrate 12, the electrodes 14, 16, and the deposited material 18. The biosensor 10 further includes an electrical connection 22. The electrical connection 22 may include, for example, a plurality of contacts for delivering an electrical signal to the active area 20 (and receiving the electrical signal back from the active area 20). The electrical connection 22 may include, for example, a plurality of electrical contacts 24. The electrical connection 22 may be formed on a plug-in tab 26 for being inserted into a corresponding slot on an electronic reader.

The biosensor 10 may further include a processing area 28. The processing area 28 may include components such as an RFID tag 30 and an antenna 32. The RFID tag 30 and antenna 32 may enable the biosensor 10 to store information and communicate via one or more data processing functions. In some embodiments, the biosensor 10 may store information, such serial numbers, batch numbers, date information, real-time location information, etc. via the processing area 28 components. Data stored on the IC on each disposable chip: (un-encrypted) may further include serial number, time and date of manufacture; (encrypted using RSA or elliptical curve) manufacture time and date (again), index number of antigen/antibody on each circuit, HemBox software compatibility, expiration date and tamper resistant encrypted data. The antenna 32 may form a holding area for the biosensor 10 for a user to safely handle the biosensor 10.

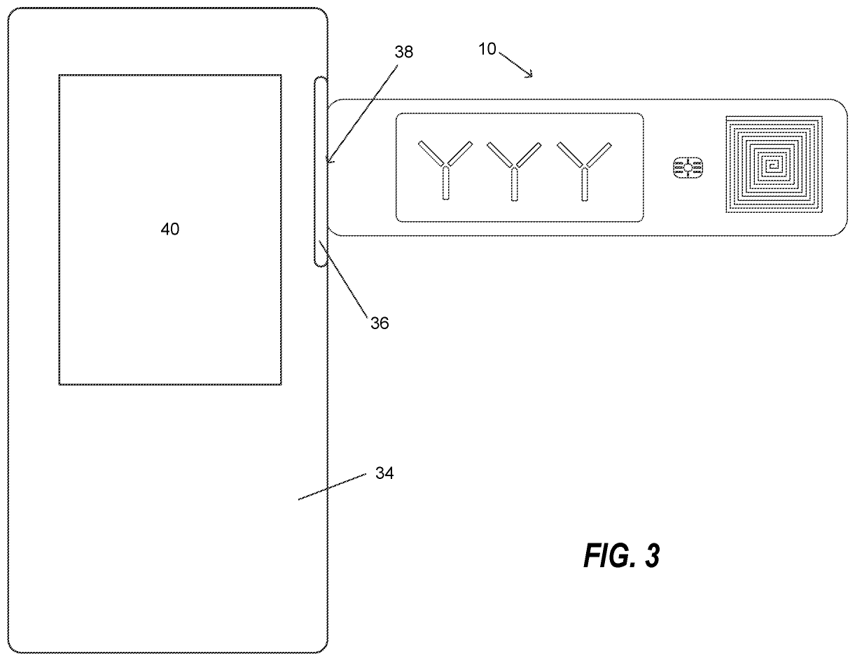

FIG. 3 is a perspective view of an electronic reader 34 for receiving the biosensor 10. The electronic reader 34 may include a slot 36 for receiving the electrical connection 22 (e.g., the tab 26) of the biosensor 10. Insertion of the biosensor 10 completes a circuit 38 within the electronic reader 28. The electronic reader 34 may further include a user interface 40 for outputting information to a user. In some embodiments, the reader 34 may provide signals to a user interface not present on the actual reader 34, e.g., via Bluetooth (or other communication means) to a monitor or other display.

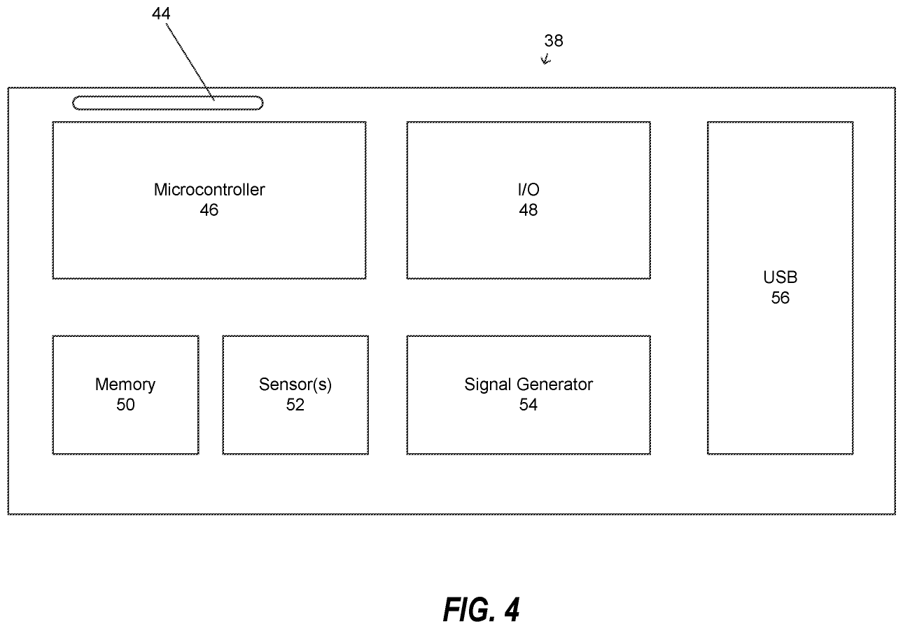

FIG. 4 is an exemplary embodiment of the circuit 38, such as a circuit board with computing components for providing a signal to the biosensor 10 and receiving an return signal to test a sample placed on the biosensor 10. In an exemplary embodiment, the circuit 38 includes a contact 44 which may be an electrical contact for interacting with the electrical connection 22 of the biosensor 10 to complete a circuit that includes the electronic reader 34 and the biosensor 10.

The circuit 38 may also include computing components including, but not limited to, a microcontroller 46, one or more I/O devices 48, a memory or other storage component 50, one or more sensors 52, a signal generator 54, and a USB or other communication hub 56. The computing components are exemplary and may be replaced with other components to execute disclosed embodiments for testing a sample via the biosensor 10.

The microcontroller 46 may be a processing device configured to monitor and control components of the circuit 38, such as to perform setup, testing, and output processes via the electronic reader 34. The I/O device(s) 48 may include the display 40, for example, to obtain input and provide output to and from a user. The memory 50 may store software instructions that, when executed by the microcontroller, perform one or more disclosed processes. The sensors 52 may include an voltage divider, resistance sensor, impedance sensor, or other device configured to determine a value associated with an electrical property at one or more locations on the biosensor 10. The signal generator 54 may be configured to generate an AC electrical signal for delivery to the biosensor 10. The USB port 56 may be a connection element for receiving power and providing data exterior to the electronic reader.

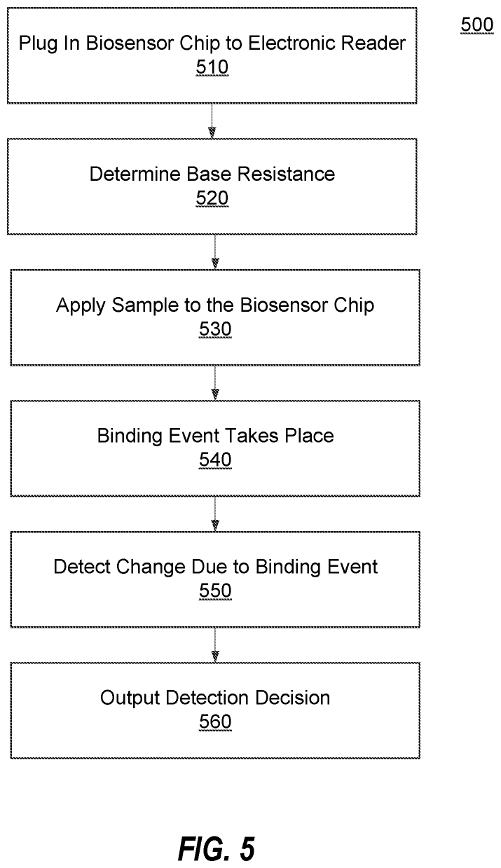

FIG. 5 is an exemplary process 500 for detecting a target biologic using a biosensor 10 and electronic reader 34. The biosensor 10 is manufactured to include a detecting biologic. The detecting biologic may be selected based on a target biologic that is desired to be detected. For example, the detecting biologic may be an antibody for detecting an antigen (e.g., bacteria) target biologic.

In an exemplary embodiment, a process for preserving the detecting biologic in the material 18 includes applying the preservative solution to a substrate in combination with carbon nanotubes and the detecting biologic. For instance, the method may include reconstituting an antibody or antigen (e.g., the detecting biologic) as instructed by the manufacturer. For example, the process may include reconstituting the antibody in water and then diluting from 0.1 to 0.5 mg/ml in preservative solution (e.g., HemSol Antibody buffer). To put the antibody on the chip, in one embodiment, a user may dilute to from 5 to 500 ug/mL in HemSol™ antibody buffer before use and put from 1 to 10 uL of this on the well of the chip. The user may then allow the antibody solution dry at room temperature (without washing). The user may then test again to determine if wells are in range.

In some embodiments, a CNT deposition process may be performed to deposit the detecting biologic together with CNTs in the gaps between electrodes on each biosensor chip. In an exemplary embodiment, the CNTs are deposited (with antibodies or other detecting biologics conjugated to them) by using a drop from a micropipette with a mixture that contains the CNT. To increase the effectiveness of the biosensor, the CNTs are aligned across a gap (20 to 100 microns wide) with conductive gold contacts on each side. In an exemplary embodiment, the CNTs are aligned through a process called AC dielectrophoresis (DEP), such as a process as disclosed in “Semiconducting Enriched Carbon Nanotube Aligned Arrays Of Tunable Density And Their Electrical Transport Properties” by Sarker et al. (July 2011), which is herein incorporated by reference. In an exemplary process, a system generates an AC signal of 500 KHz to 5 MHz at 5V to 15V peak to peak and the drop is allowed to dry while the AC signal is applied across the gap through the gold contacts. Once dried, the CNT is stuck in place. By using a metallurgical microscope with reflected illumination, a user can broadly observe the level of the CNT concentration. The process is then repeated and the concentration of CNT is adjusted in the drop, as well as the peak to peak voltage. This process is continued until a specified ohmic range (100k to 1M ohm) is obtained.

The preservative solution, e.g. HemSol™ preserves the conjugation of the CNTs and the detecting biologic, e.g. antibody. In this instance, the CNTs are hydrophobic and the antibody (proteins) are not. The preservative solution facilitates and maintains the conjugation of the two despite this difference.

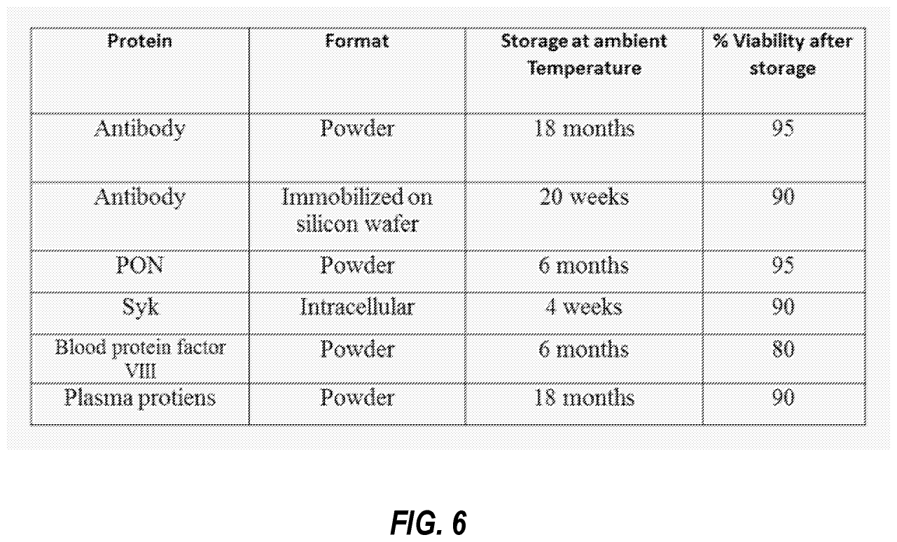

FIG. 6 is a table depicting several types of proteins and formats using the preservative solution (e.g., HemSol™) showing enhanced shelf-life. The use of the preservative solution (e.g., HemSol™) extends antibody-on-CNT shelf life from ˜4 hours to +18 months. The antibody-on-CNT requires about 15-30 seconds to rehydrate before the antibody on CNT is ready for use. Once a sample is placed, it takes less than 2 seconds to detect Ab-antigen binding when used with the sample carrier and sample reader as described below.

In an exemplary embodiment, the deposited material includes, for example, carbon nanotubes (NanoC-Purchased from NanoC) in deoxycholide, graphene stock in 0.1 to 1.0 mg/ml in 0.1 to 10.0% sodium dodecyl sulfate (SDS), and polymer at saturated condition in water (conducting polymers such as polyacetylene; polyphenylene vinylene; polypyrrole; polythiophene, polyaniline, and polyphenylene sulfide may be used). Here, the conducting polymer, ADS2000P, was purchased from American Dye Source Inc. Shake it up and then wait for the polymer to settle before use. Take the top to avoid the precipitate.

Then, a user mixes the ingredients to make a working solution. For example, 10 to 1000 uL of Graphene+10 to 1000 uL of NanoC may be used to create a nano solution mixture. The Nano working solution is 100 uL of the nano solution mixture+100 uL of the saturated polymer. The sample carrier comprises multiplexed circuits having a 1 to 50 um gap width and from 1 to 300 circuits per chip. A piezo-electric head on an X-, Y-, and Z-axis gantry may then be guided by a multiple cameras to deposit the CNT-Ab mix on the gaps and nearby traces of the open circuits.

The sample carrier chip is designed with multiple circuits for testing the variation in resistance of carbon nanotubes coated with detecting biologics (e.g., antibodies or nucleic acids). These circuits consist of two terminals separated by a 1 to 50 um gap. On an exemplary chip, three different interconnected metal layers are used to form a matric of open circuits that are connected by the CNT-Ab mix.

In process 500, the biosensor chip 10 is inserted into the electronic reader 34 (step 510). This completes the circuit and enables the electronic reader to deliver a signal to the biosensor chip 10. The electronic reader 34 may then determine a base resistance (and/or impedance) at each electrode site (step 520). For instance, the electronic reader 34 may deliver a pulsed signal at a selected frequency (e.g., 30 Hz) to determine an impedance curve that sets a baseline at each electrode site (e.g., for each detecting biologic on the biosensor 10). The electronic reader 34 may store the baseline reading and map each reading to an electrode site for future comparisons/tracking of multiple detection molecules.

In an exemplary embodiment, the microcontroller 46 has an analog multiplexer (mux) that sends signals to the various terminals of the circuits formed on the biosensor 10. The micro controller polls the circuits using an X-coordinate mux and Y-coordinate mux. On any given circuit, the micro controller using an alternating current (with no DC bias) sends a full current cycle across every circuit it polls.

A user may then apply a sample to the biosensor chip 10. For example, a user may place a drop of blood or saliva on the active area (step 530). The sample material impinges on the deposited material 18 on the biosensor and one or more binding events may take place (step 540). Carbon nanotubes, which bridge the 1-100 um gap, act as a field-effect transistor. When bacteria, which are negatively charged, connect to the carbon nanotubes via conjugated antibodies, these microorganisms act collectively as a FET gate and thus impede current flow (increase resistance). As a micro controller polls a circuit, it will register ˜60% delta in resistance. Before bacteria bind to the carbon antibodies, these circuits have a resistance of 10K ohms to 800K ohms. Thus, resistance demonstrates the presence of the bacteria. In the process 500, the electronic reader compares the resistance readings after the sample is applied to the baseline readings to detect the change and thus the binding event (step 550). The electronic reader is configured to determine the binding event that was detecting and provide output (e.g., target biologic X was detected) (step 560). In another example, where no binding event takes place, the electronic reader may output a negative test result for the associated target biologic.

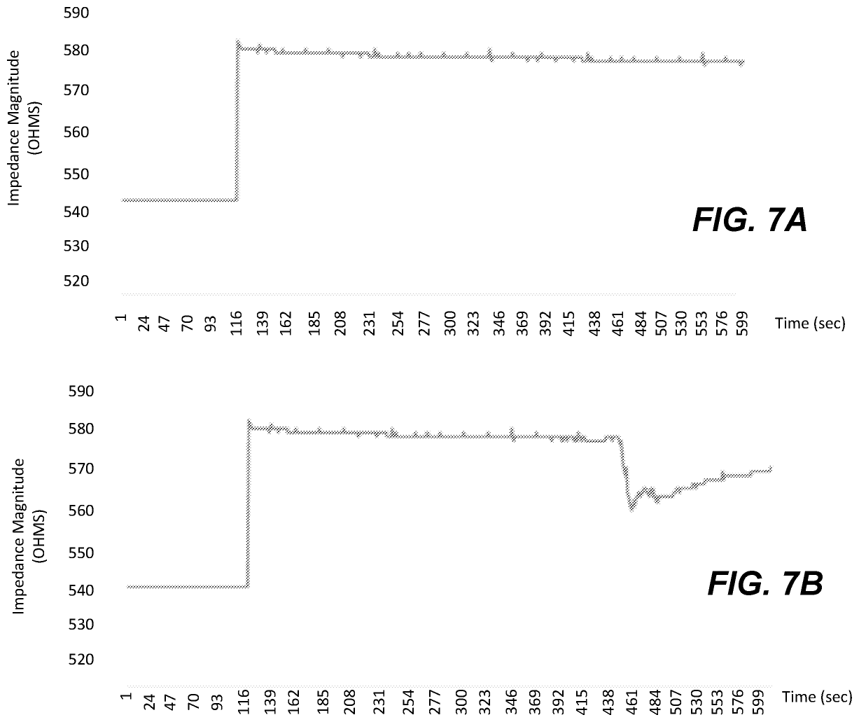

FIG. 7A is a graph of an impedance output that may be detected without a sample applied based on an AC signal pulse. The impedance spikes at the signal start and levels off at a base value. FIG. 7B is a graph of an impedance output that may be detected when a sample has been applied and a binding event has occurred, resulting in an impedance dip as a result of the binding. The electronic reader 46 may be configured to store output maps such as those shown in FIGS. 7A-7B and include information that indicates which graph is associated with a binding event and which is associated with no binding event.

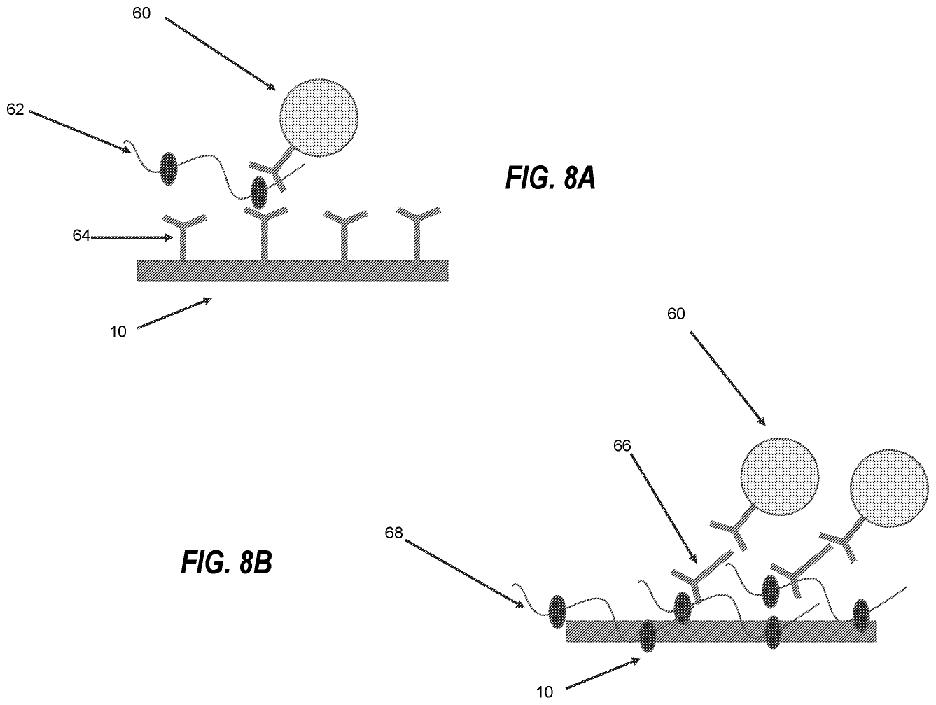

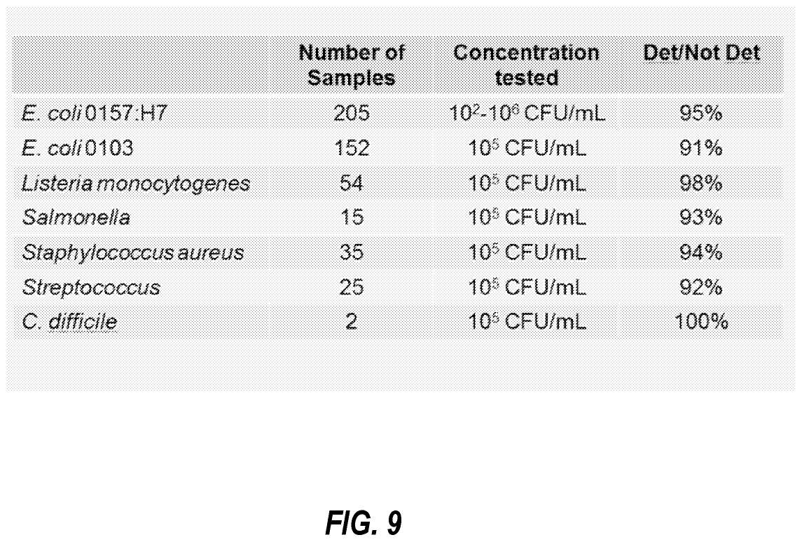

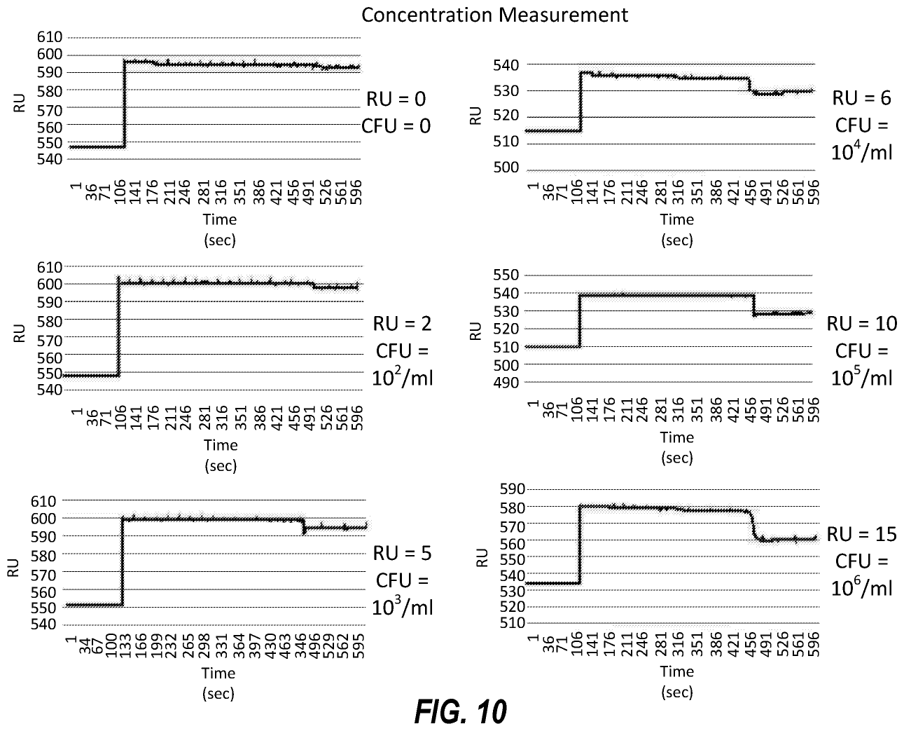

In some embodiments, the binding events that occur between a detecting biologic and a target biologic may be sufficient to produce a detectable impedance change. This may be considered label-free detection of target biologics. In some embodiments, labeled detection may be used to further enhance the potential for a detectable change in impedance at a binding event. For example, for proteins, cells or cell fragments (samples) that do not carry enough charge to generate a signal impedance on the circuits, antibodies that have been conjugated to gold nanoparticles (Ab-GNP) may be introduced into a sample to amplify the impedance potential of the samples so that the electronic reader can pick up the change in impedance. FIG. 8A is a depiction of a conjugated antibody 60 in a sample binding to a target antigen 62 in the sample and also binding to a detection antibody 64 on the biosensor 10 (e.g., the detection antibody 64 may be in a material 18 deposited on the biosensor 10). FIG. 8B is a depiction of an alternative embodiment in which a conjugated antibody 60 in a sample, together which non-conjugated antibodies 66 in the sample, bind to antigens 68 on the biosensor 10 (e.g., the detection antibody 64 may be in a material 18 deposited on the biosensor 10).

An example testing chip that was produced and used for testing, as well as the testing results, will now be described. To make these chips, a silicon wafer is coated with a 100 to 500 nm layer of SiO₂. On top of this a foundry applies a network of 5 to 15 nm of chrome and 20 to 40 nm gold circuit traces. The key features of this system are: multiple circuits for testing for a delta in current resistance across a 1-50 um gap. Carbon nanotubes (CNT) with antibodies bridge the two circuit terminals. Once an antigen, RNA or DNA binds with its CNT-bound compliment, the resistance across the gap will vary by ˜60% within a fraction of a second. An exemplary process includes the treating the chip surface with polysiloxanes, spray a layer of polysiloxanes on chip surface, wait for 5 min at room temperature (RT) and then wipe dry. Further, a user may apply 1-10 uL of nano working solution to the wells of the chip, wait until dryness at RT, soak the chip in water for about 30 min to wash out excess nano working solution, shake off water and leave chips at RT to dry, and test to determine which wells are in range.

In one embodiment for attaching the detecting biologics, a user may make a preservative solution for the detecting biologic (e.g., antibody “Ab”) by making 1:100 dilution of HemSol™ 2× in 1×PBS or similar buffer—this is the HemSol Ab buffer to dilute and preserve Ab. Exemplary steps may include receiving Ab and reconstitute as instructed by the manufacture, reconstituting Ab in water then dilute to 0.1-1 mg/ml in HemSol™ Ab buffer. This is the stock solution for Ab, and diluting to 10-100 ug/mL in HemSol™ Ab buffer before use. Then the user may put 1-10 uL of the resulting solution on the well of the chip, allow the Ab solution dry at RT (Do not wash). The user may then test again to determine if wells are in range. The working range for testing may be 0.5 to 1.5 volt, with a signaling frequency 500 Hz to 6 kHz with no DC bias, a signal generation via a PWM through a pi filter or a digital-to-analog converter, and an analog-to-digital signal conversion of signal passed through chip. Curve-reading software may be implemented to read an impedance curve. In testing, the sample reader used a successive approximation register (SAR) A-to-D converter to digitize and records sample carrier impedance changes caused by bacteria binding to antibodies on a carbon nanotube field-effect transistor.

The sample reader polled from 1 to 300 circuits every 15 to 45 times per second reading the resistance of every circuit using an AC current. This resistance measurement is used by the curve-reading software to determine if bacteria does or does not bind to antibodies on a given circuit.

A program running on the sample reader, which filters signal noise and uses a moving average and various statistical functions, looks at the digital stream of values to find a binding curve pattern. If a binding pattern is found within a prescribed period of time, the sample reader returns “detected” and if it is not found within the allotted time, a “not detected” is displayed on the screen of the device.

Heat killed E. coli O157:H7, and anti-E. coli O157:H7 polyclonal antibodies were purchased from Kirkegaard and Perry Laboratories Inc. (Gaithersburg, MD, USA). A stock solution of 10⁹ colony forming units/mL (CFU/mL) E. coli O157:H7 was made using diH₂O. A 1 mg/mL stock solution of the antibody was made in diH₂O. Dimethyl formamide (DMF), 1-pyrene butanoic acid, 1-Pyrenebutyric acid Nhydroxysuccinimide ester, 1-pyrene butanoic acid hydrazide,1-Ethyl-3-(3-dimethylaminopropyl) carbodiimide hydrochloride (EDC), N-Hydroxysuccinimide, sodium metaperiodate (NaIO4), 2-(N-morpholino)ethanesulfonic acid.

(MES), dimethyl sulfoxide (DMSO), and ethanol were purchased from VWR. GlycoLink Immobilization Kit and Zeba desalting columns were purchased from Pierce. Dextran-70, Trehalose, Mannitol, and Dextrose were purchased from Sigma. All chemicals were used as received.

Single-walled carbon nanotubes (CNTs) containing about 70% conducting nanotubes were purchased from Carbon Nanotechnologies Inc. 92 sample-well CNT-FETs were manufactured on standard 4 inch silica semiconductor wafers by NanoPlatform Inc. using standard photolithography and lift-off process. Briefly, an octadecyltrichlorosilane monolayer pattern on silicon oxide wafers was generated by first patterning AZ 5214 photoresist via standard photolithography, dipping the wafer in the octadecyltrichlorosilane solution (1:500, v/v in hexane) for 3 min, and removing the PR patterns using acetone. The CNT solution was prepared by dispersing purified CNT in 1.5% SDS with ultrasonication for 7 h (concentration 0.04 mg/mL). Then, 2 uL of the sonicated CNT pipetted on the surface of the patterned silicon oxide wafer and allowed to dry for 30 minutes at room temperature. The wafer was rinsed thoroughly with dH₂O dried at room temperature. This step allowed CNT to be adsorbed selectively onto bare SiO₂ regions on the wafer.

A desiccation buffer is described in U.S. Pat. No. 9,943,075, which is hereby incorporated by reference. An exemplary buffer (HemSol) was prepared by adding a ratio of low molecular weight sugars (7% Trehalose, 2% Mannitol, and 2% Glucose) and high molecular weight sugar Dextran (6%) in PBS buffer pH 7.4.

Solutions of antibodies and reagents were incubated on the wafers in a 37° C. oven to promote dry absorption of antibodies onto the circuits. Briefly, 2 μL of 10 μg/mL of the polyclonal anti-E. coli O157:H7 antibody in either PBS or HemSol was incubated with the circuits for 30 minutes. After the incubation, wafers were ready to be tested.

For the bacteria detection assays, a baseline impedance value for each circuit was first obtained by applying 2 μL PBS to the circuit and recording the signal for approximately 30 sec. Then, 2 μL of the E. coli in PBS, at concentrations of 10⁶ CFU were carefully admixed on the circuits and changes in impedance were measured for up to 3.5 additional minutes. The impedance value for each measurement was normalized to the corresponding baseline value. Each solution of the E. coli was measured at least in quadruplicate using a fresh circuit for each measurement. A source/drain bias of 100 mV was maintained throughout the measurements of the electrical signal and the pulse width was 1 sec. The electrical properties of the samples binding the CNT-FET were measured using a low current measurement system (MediSourcePlus Inc.) that makes electrical contact to the source and drain electrodes of the CNT-FET. The transfer characteristics of this circuit design typically observed electronic transfer changes from 20 to 10 nanoamperes before and after the antibody immobilization on the CNT-FET circuits when Vds and VG are 0.1 and −0.1 volt, respectively.

A baseline impedance value for each circuit was first obtained by applying 2 μL PBS to the circuit and recording the signal for approximately 30 sec. For E. coli samples, 2 μL of the E. coli in PBS, at concentrations of 106 CFU were carefully admixed on the circuits and changes in impedance were measured for up to 3.5 additional minutes. A calculation was performed by taking impedance changes for samples contained E. coli and divided by impedance changes for baseline. Each data point represented the average of 4 readings.

Determination of Optimal Coating Concentration for E. coli O157:H7 Polyclonal Antibody on CNT-FET Circuits.

| Antibody | | | |
|---|---|---|---|
| Concentration | Impedance | | |
| (μg/mL) | Baseline* | E. Coli** | Normalized |
| 0.001 | 0.9 | 0.91 | 1.01 |
| 0.01 | 1.12 | 1.12 | 1.00 |
| 0.1 | 1.14 | 1.13 | 0.99 |
| 1 | 0.98 | 0.99 | 1.01 |
| 10 | 0.98 | 1.25 | 1.27 |
| 100 | 0.96 | 0.98 | 1.02 |

E. coli O157:H7 polyclonal antibody diluted in PBS at various concentrations were incubated with the CNT-FET circuits for 30 minutes. After the incubation, circuits were tested for E. coli binding to the antibodies. Results from this data showed that optimal concentration for antibodies to be used on the CNT-FET circuits was about 10 μg/mL.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

The system and processes of the figures are not exclusive. Other systems, processes and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112, sixth paragraph, unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

1.-20. (canceled)

21. A method of preparing a biosensor chip for detecting a target biologic, the method comprising:
providing a substrate comprising at least one electrode region adapted for detecting the target biologic:
contacting at least one detecting biologic with a preservation solution comprising at least one membrane-penetrable sugar and at least one membrane-impenetrable sugar;
depositing the at least one detecting biologic and the preservation solution on an active area of the substrate proximate the at least one electrode region; and
drying the at least one detecting biologic and the preservation solution on the substrate to a final moisture content of about 5% to about 95%, thereby forming a biosensor chip in which the at least one detecting biologic is preserved for subsequent detection of the target biologic.

22. The method of claim 21, wherein the membrane-penetrable sugar comprises trehalose.

23. The method of claim 21, wherein the membrane-impenetrable sugar comprises dextran.

24. The method of claim 21, wherein the final moisture content is from about 15% to about 40%.

25. The method of claim 21, wherein the final moisture content is from about 20% to about 25%.

26. The method of claim 21, further comprising contacting the at least one detecting biologic with a fixative agent prior to the drying step.

27. The method of claim 26, wherein the fixative agent is glutaraldehyde, paraformaldehyde, or paraldehyde.

28. The method of claim 21, wherein the preservation solution further comprises at least one anti-microbial agent.

29. The method of claim 21, further comprising vacuum desiccating at a temperature from about 0° C. to about 40° C. for about 1 hour to about 24 hours.

30. The method of claim 29, wherein the vacuum desiccation is performed for about 3 hours at a temperature from about 32° C. to about 34° C.

31. The method of claim 21, further comprising storing the dried biosensor chip in a vacuum-sealed container.

32. The method of claim 21, wherein the substrate comprises a silicon-based material having a hydrophobic carbon-based layer comprising at least one carbon allotrope or other two-dimensional material, and wherein the preservation solution prevents removal or denaturation of the detecting biologic from the hydrophobic layer during the drying step.

33. The method of claim 21, wherein the at least one detecting biologic is an antibody, an antigen, a cell, a cell fragment, a virus, a parasite, a nucleic acid, a carbohydrate, a lipid, a protein, or a combination thereof.

34. The method of claim 21, wherein the preservation solution further comprises an antioxidant.

35. The method of claim 21, wherein the preservation solution further comprises at least one salt, a buffer, or a chelating agent.

36. The method of claim 21, further comprising depositing one or more conductive nanomaterials on the substrate prior to or concurrent with depositing the at least one detecting biologic.

37. The method of claim 36, wherein the conductive nanomaterials include at least one carbon allotrope or other two-dimensional material, or a conducting polymer, and the at least one detecting biologic is conjugated thereto.

38. The method of claim 21, further comprising rehydrating the at least one detecting biologic prior to use by contacting the dried biosensor chip with water or saline.

39. The method of claim 21, wherein the method is performed without lyophilization, freeze-drying, or oven-drying steps.

40. A method of preparing a biosensor chip for detecting a target biologic, the method comprising:
providing a substrate comprising at least two electrodes defining an active region, wherein the substrate includes a hydrophobic carbon-based layer including at least one carbon allotrope or other two-dimensional material;
preparing at least one detecting biologic by contacting the at least one detecting biologic with a preservation solution comprising:
at least one membrane-penetrable sugar selected from trehalose and glucose,
at least one membrane-impenetrable sugar selected from dextran having a molecular weight of about 40,000 Daltons or more,
at least one anti-microbial agent, and
at least one antioxidant;
depositing the at least one detecting biologic and the preservation solution onto the active region of the substrate so as to immobilize the at least one detecting biologic between the two electrodes; and
drying the at least one detecting biologic and the preservation solution to a final moisture content from about 5% to about 95%,
wherein the preservation solution maintains the detecting biologic in place on the hydrophobic carbon-based layer without substantial denaturation or removal during drying, and
whereby the dried biosensor chip exhibits an extended shelf-life at ambient temperature for detection of the target biologic upon rehydration.
